(19) United States
(12) Patent Application Publication
(10) Pub. No.: US 2010/0249884 A1
(43) Pub. Date: Sep. 30, 2010

(54) Medical Device and Method for Operating the Same

(76) Inventors: Mathew A. Chandy, Bangalore (IN); Jürgen Schwarzmann, Eggolsheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/730,433

(22) Filed: Mar. 24, 2010

(30) Foreign Application Priority Data
Mar. 24, 2009 (DE) 10 2009 014 621.0

(51) Int. Cl. A61N 1/08; G06F 15/16

(52) U.S. Cl. 607/60; 709/217

(57) ABSTRACT

In the case of a medical device with several electronic components, a possibility must be provided for enabling the components to be rapidly matched to each other, in order to guarantee the functional correctness of the medical device. In particular in the case when, for example, an item of operating software is modified for one individual component only, it can occur that this component no longer interoperates with the other components in a desired way. In this context, at a particular point in time, for each of the electronic components of a apparatus data for the component is copied and the copied data is stored outside the storage facility of the component, making it possible at a later point in time, which can be defined, for the copied data for each component to be stored in its storage facility.

### CROSS REFERENCE TO RELATED APPLICATIONS

This application claims priority of German application No. 10 2009 014 621.0 filed Mar. 24, 2009, which is incorporated by reference herein in its entirety.

### FIELD OF THE INVENTION

The invention relates to a method for operating an apparatus with electronic components, in particular of a medical device, a method for integrating an electronic component into such an apparatus, and to a medical device with a communication facility, a central storage device and at least one further electronic component in accordance with the claims.

### BACKGROUND OF THE INVENTION

Large medical devices for investigating or operating on patients may incorporate several computers, microprocessors or control units which, within the medical device, monitor and control individual parts of the medical device. By means of a communication network inside the device, these individual electronic components of the medical device can exchange items of data, by which means the components enhance each other's functionality. An example of such a complex medical device is the applicant company's Artis Zee multi-purpose system.

During the operation of such a medical device, it is consistently necessary to carry out maintenance work on the individual components. For example, it can happen that the manufacturer of a control unit built into the medical device makes available an improved version of a control program, or that a program for a computer which is built into the medical device is replaced by a version of it which has been further developed.

Such maintenance can also change such functions of a component which are used by another component of the medical device. Thus, for example, a computer makes available items of control data which are called up by a control unit and, for example, used to control an x-ray unit in the medical device.

When making a change to such functions of a component, which are used by other components, it is always necessary to avoid a component being no longer able, after a maintenance action, to receive items of data from another component, because it is unable to call up the data appropriately to the other component's new mode of operation. It is equally essential to avoid the interoperation between the components being disrupted after maintenance work, e.g. by an incorrectly installed program.

Malfunctions which arise in conjunction with an incompatible or faulty mode of operation by individual components in a medical device would, namely, very probably only become apparent when a component of the medical device wishes to use the doubtful function. However, at this point in time it would then be difficult and time-consuming to find out exactly which function is causing the malfunction. Also, it would then often no longer be clear which component must be adjusted in order to eliminate the malfunction, without in doing so causing malfunctions in other places.

Similar problems arise for a medical device if a component is defective and must be replaced by a new component. It is then very time-consuming to match the new component to the other components in the medical device in such a way that the medical device as a whole returns to fault-free functioning.

The down times of the medical device arising in conjunction with fault location and the mutual rematching of the components of a medical device are unacceptable. Due to the high procurement cost, such a device is often planned for investigations or operations around the clock. It may also be that such a device must, for example, be constantly available for emergencies.

In relation to a reconstruction of data which has been deleted from a storage facility in a computer against the user's wishes, the known method is to store a copy of important data items from the storage facility at regular intervals in a second storage facility, where they are protected against any intervention by the user. Such a copy is also referred to as a backup of the data. If needed, the copied data can then be made available to the user. This procedure is also called a restore or reconstruction.

### SUMMARY OF THE INVENTION

The object of the present invention is to create the possibility, for an apparatus, in particular a medical device with several electronic components, of being able rapidly to match a plurality of the components to each other in such a way that the functional integrity of the apparatus is guaranteed.

This object is achieved by a method of operating an apparatus, by a method for integrating an electronic component, and by a medical device as claimed in the claims.

With the method in accordance with the invention for operating an apparatus, it is possible to operate an apparatus in which at least two electronic components interoperate, and a method of working for each component which determines the interoperation is defined by data items which are stored in a storage facility of the component in each instance. The apparatus is preferably a medical device. If the electronic component in this case is a computer, the data items might then be, for example, the operating software and configuration data on the computer's hard disk. However, the data items might also be parameter values for a configurable control unit, e.g. for a digital x-ray device or a positionable patient table.

In accordance with the method, when a particular configuration state exists for the apparatus, the data for a component is copied for each of the components and the copied data is stored outside the component's storage facility, thus making it possible at a definable later point in time to store the copied data items for each component into the storage facility. Here, a configuration state of the apparatus is determined by the totality of the data items which are stored in the components' storage facilities and by which the ways of working of the individual components are defined. Overall, the mode of operation of the apparatus itself is also defined by the totality of the data items. Correspondingly, the configuration state is modified by storing new data, e.g. new software or new parameter values, in at least one storage facility of one of the electronic components.

This method in accordance with the invention gives the advantage that the apparatus can at any time be put into a state in which the apparatus has already been once before and in which the components function in a desired way. For this purpose, according to the method a point in time is first determined at which the apparatus is in a desired state. At this point in time, the data items are then read out from all the components, and saved. Indirectly, therefore, the state of the apparatus itself is thereby recorded. This state can be restored by writing the saved data back into the storage units of the individual components of the apparatus.

Here, the copying of the data items need not take place at exactly the one and same time. The copying of the data can take place over a time interval during which the apparatus is unchanged in the particular configuration state which is to be made restorable by the copying of the data items and the storage of the copied data items.

An advantageous development of the method is given by the following additional steps:
detect an operating state of the apparatus,
depending on the operating state which has been detected, if necessary interrupt the interoperation of all the components and for each component store the copied data items in the storage facility.

By this development of the method it is possible to achieve the effect that the apparatus functions once more in an acceptable way after the apparatus had got into an undesired operating state. The detection of the operating state will preferably take place automatically.

However, it is very difficult in the case of a complex system, consisting for example of several computers, to automatically detect an operating state of the device. Here, an advantageous development of the method results if the actuation of an operating element effects for all the components the storage of the copied data items in the storage facility. By this means, an operator is afforded in an advantageous way a possibility for manually restoring a state of the apparatus represented by the copied data items.

A further advantageous development of the method results if the copied data items for the components are stored externally in a common central store. This central store makes available in an advantageous way a facility in which are stored all those items of data which are necessary for putting the apparatus into a particular state.

The method is, furthermore, developed in an advantageous way if a memory map of a storage medium in the storage facility is created for the purpose of copying the items of data. Preferably, the storage facility concerned will be a hard disk. A corresponding memory map is then referred to as a “disk image” (hard disk storage map or hard disk map). This development of the method gives the advantage that it is possible to copy the data items independently of the structure in which the data items are held on the storage medium. Apart from this, it is advantageous that the data items can be written back even if the storage medium has not yet been prepared, for example by formatting, for having data written to it.

A further advantageous development of the method results if the way in which the components work can be defined in addition by conveying to the components instructional data, via a communication network, and in order for each component to effect storage of the copied data in the storage facility instructional data is initially conveyed to the component, defining the mode of operation of the component in such a way that the component stores the copied data in its storage facility.

This gives the advantage that the writing back of the copied data items into the individual components' storage units can be controlled centrally. For example, a component with which the apparatus is provided might be a central control unit which, if a fault occurs, conveys the instructional data to the other components and then prompts the components prepared in this way to call up the copied data from a central store and to store it in the storage facility of the component concerned.

A further advantage results when the data items or the instructional data include program codes. This makes it advantageously possible also to use the method for any apparatuses in which a computer network comprising several computers is a component of the apparatus.

Finally, the method is advantageously developed in that the step which includes the copying of the data and the storage of the copied data is
a) repeated at prescribed points in time, or
b) carried out after or before a change, in particular each change, is made to data in at least one of the storage facilities.

In that the state of the apparatus is, in accordance with sub-step a), regularly detected and saved, and indeed preferably automatically, data items will be saved while the apparatus is in different states. Preferably, several sets of detected data will be stored, where a set of detected data includes the data items detected at a particular point in time. If a malfunction of the apparatus occurs the different sets of recorded data then advantageously make available a choice of states into which the apparatus can be put. Automatic saving of the data items achieves the additional effect that the saving of the data does not require an operator to initiate it. Sub-step b) gives the advantage that any change to the data in the components' storage facilities can be reversed at any time.

A solution to the object is also given by a method in accordance with the invention for integrating an electronic component into a medical device. This method can be used for an electronic component for which a method of working can be defined by data where the data is stored in a storage facility for the component. Here again, an example of such a component is a computer, on which software can be stored on the hard disk as the storage facility. The method includes the steps:
linking the component to the medical device,
transmitting the data from a central store on the medical device to the component and store the data in the component's storage facility.

This method in accordance with the invention gives the advantage that a new component can be matched to the other components of the apparatus in a simple way.

The method is developed in an advantageous way in that the data is copied from another electronic component's storage facility and is stored in the central store, and the other component is removed and replaced by the component which is to be integrated into the device.

In an advantageous way, the new component of the apparatus which is to be integrated thereby automatically provides those functions which were made available by the other components before its removal. In other words, the effort involved when a defective component is replaced by a new component can be reduced.

This is especially advantageous when the data includes program codes. It even permits a computer in a system to be exchanged without the new computer needing to be equipped in some special way with software for operation within the apparatus.

The other developments, described in connection with the inventive method for operating an apparatus, are of course also transferable in an appropriate way to the inventive method for integrating an electronic component. Correspondingly, the advantages described in connection with the development concerned, of the method for operating an apparatus, also apply.

The invention also covers a medical device with a control unit, a communication facility, a central storage device and at least one other electronic component. In the case of the device in accordance with the invention, a mode of operation of the component can also be defined by data if this data is stored in a storage facility for the component. In addition, the component is linked to the communication facility via one of several connections. In addition, data to define different modes of operation of a component are stored in the device, in the central storage device. In the case of the inventive medical device, the control unit is embodied to effect the transmission of data, defining a method of working, from the central store to the component, as a function of which one of the connections connects the component to the communication facility.

In the case of the medical device in accordance with the invention, the mode of operation of the other electronic components can advantageously be adjusted virtually automatically. By reference to the connection via which the component is connected to the communication facility, the functions which the component should provide in the device are recognized. Correspondingly, its mode of operation is defined by storing appropriate data in the component's storage facility.

With the inventive medical device, it is also advantageously possible to carry out the two inventive methods and their developments. Correspondingly, the advantages described in connection with the method concerned and its developments also result for the medical device if it is designed to carry out the appropriate form of embodiment of one of the methods.

### BRIEF DESCRIPTION OF THE DRAWINGS

The invention is described in more detail below, by reference to the following exemplary embodiments. Shown in this connection are:

FIG. 1 a plan of a communication network as it is constructed from computers installed in a medical multi-purpose system, in a medical multi-purpose system which conforms to one form of embodiment of the inventive medical device, and

FIG. 2 the plan as shown in FIG. 1 highlighting operations, which take place in the integration of a new computer into the multi-purpose system, in accordance with one form of embodiment of the inventive method for integrating an electronic component into the multi-purpose system.

### DETAILED DESCRIPTION OF THE INVENTION

The exemplary embodiments represent preferred forms of embodiment of the invention.

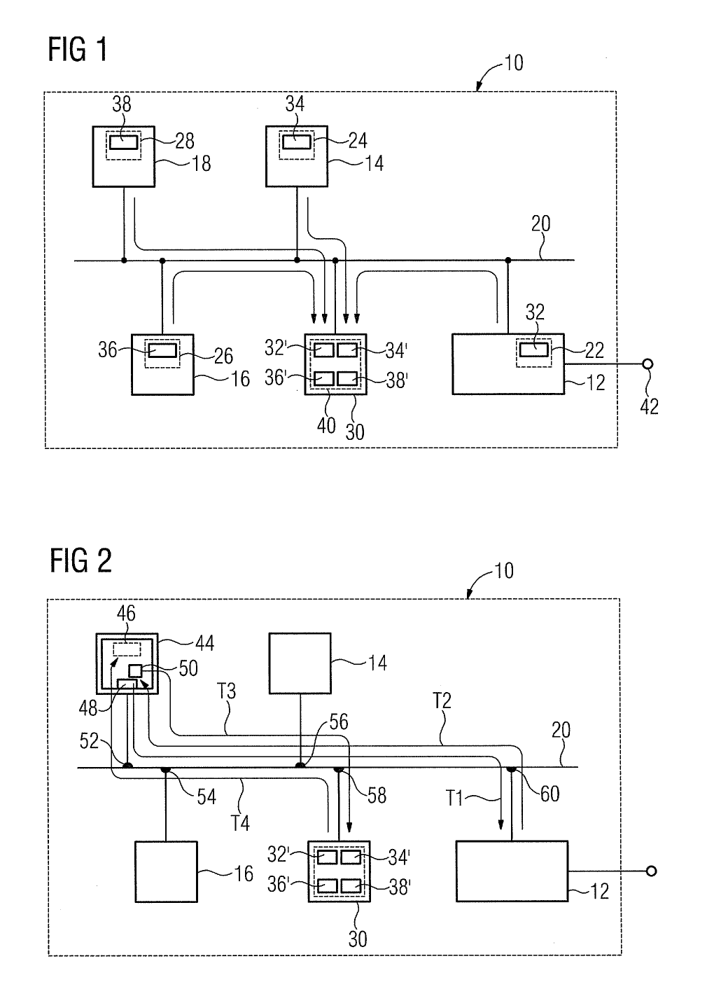

FIG. 1 illustrates a plan for a medical multi-purpose system 10, where several computers 12, 14, 16, 18 are components of the multi-purpose system 10. In the multi-purpose system 10, the computers 12, 14, 16, 18 are linked together to form a local area network (LAN). For this purpose they are connected to a common network facility 20 of the multi-purpose system 10. In the present case the network facility 20 conforms to the Ethernet standard.

In the example shown in FIG. 1, the computer 12 provides a central control system for the multi-purpose system 10. An operating state, for example, of the multi-purpose system 10 can be indicated to an operator via the computer 12. Apart from this, provision can be made that the operator effects settings for the multi-purpose system 10 via the computer 12. In doing so, control instructions are conveyed from the computer 12 to the other computers 14, 16, 18. An operator can also be given the capability to install software in one of the computers 12, 14, 16, 18, via the computer 12.

The computer 14 is used to control an x-ray C-arm, not shown in FIG. 1, of the multi-purpose system 10, through which the digital medical image data can be obtained. This is then transmitted to the computer 16.

The image data obtained by means of the x-ray C-arm can then be observed by a user of the multi-purpose system 10 on a screen of the computer 16, not shown in FIG. 1. What cannot be seen from FIG. 1 is that the computer 16 is accommodated in a different room from that in which the x-ray C-arm of the multi-purpose system 10 is standing. In spite of this, the computer 16 is a component of the multi-purpose system.

The computer 18 belongs to an operating unit which is located directly adjacent to a patient table of the multi-purpose system 10, not shown in FIG. 1. Not shown in FIG. 1 is a screen for the computer 18 with a touch-sensitive surface (a touchscreen), through which the user can move the patient table and the x-ray C-arm. In doing this, control instructions are transmitted from the computer 18 to the computer 14.

The structure of the multi-purpose system just described is only chosen as an example, to enable the elements which are essential for an explanation of the invention to be better visualized in what follows.

Each of the computers 12, 14, 16, 18 of the multi-purpose system 10 provides a hard disk 22, 24, 26, 28 as a storage unit on which is stored in each case a file system with an operating system and other software for the computer 12, 14, 16, 18 concerned. The operating system and the other software make possible the functionalities of the individual computers 12, 14, 16, 18, described above. The software, including the operating system, which is stored on a hard disk 22, 24, 26, 28 represents data which defines the way in which the computers 12, 14, 16, 18 work. The operating system and the other software of any of the computers 12, 14, 16, 18 are matched to the operating system and the other software of any of the other computers 12, 14, 16, 18, so that image data and control data can be exchanged rapidly and without difficulty between the computers 12, 14, 16, 18.

Also connected to the network facility 20 is a central file server 30, e.g. an NAS (Network Attached Storage). In the file server are held files which are used by the computers 12, 14, 16, 18 which must not be lost even if, for example, one of the hard disks 22, 24, 26, 28 should become functionally incompetent. Instead of the central file server 30, it is also possible to provide for the files in the computer 12 to be held in a storage device which is designed for that purpose or to make available another computer with storage capacity appropriate for holding the files.

Stored on each hard disk 22, 24, 26, 28, in the example in FIG. 1 in a partition 32, 34, 36, 38, is the file system with the operating system it contains and the other software it contains. Each computer 12, 14, 16, 18 has transmitted a copy of the relevant partition 32, 34, 36, 38 on its hard disk 22, 24, 26, 28 to the file server 30, where they are stored as a hard disk image 32′, 34′, 36′, 38′ on a hard disk array 40 (RAID—Redundant Array of Inexpensive Discs) in the file server 30. In FIG. 1, the procedure of transmitting the hard disk images 32′, 34′, 36′, 38′ of the partitions 32, 34, 36, 38 to the file server 30 is indicated by arrows. The hard disk images 32′, 34′, 36′, 38′ can be created using software known from the prior art, which can be a component of the software installed as on the computer concerned.

The time point at which the computers 12, 14, 16, 18 create the relevant hard disk images 32′, 34′, 36′, 38′ and transmit them to the file server 30 can, for example, be defined in advance by an operator at computer 12. At the time point thus prescribed, the computer 12 then autonomously transmits an appropriate signal via the network facility 20 to the other computers 14, 16, 18, to prompt the latter to create the hard disk images 32′, 34′, 36′, 38′ and to transmit them to the file server 30. The operator can also be given the option, by the computer 12, to initiate the creation of hard disk images by directly calling up an appropriate function of some control software on the computer 12.

It is also possible to store on the file server several hard disk images of each hard disk 22, 24, 26, 28. In this case, an operator can be given an overview via the computer 12 of the images which can be called up on the file server 30.

The image data, for example obtained by means of an x-ray C-arm, can be called up from outside the multi-purpose system 10 via the computer 12, through a network connection 42 of the multi-purpose system 10. For this purpose, the image data is stored in the computer 12 on a hard disk array (RAID), not shown in FIG. 1. It is also possible to enable the storage of the hard disk images 32′, 34′, 36′, 38′, in a file server which is not directly connected to the network facility 20, via the network connection 42.

A plan of the multi-purpose system 10 described in conjunction with FIG. 1 is shown once again in FIG. 2. For this reason the same reference marks are used in FIG. 2 for those elements which correspond to elements shown in FIG. 1. Furthermore, elements which have already been explained in conjunction with FIG. 1 are not explained again.

The way in which a defective computer in the multi-purpose system 10 is replaced by a new one, and the latter is configured for operation within the multi-purpose system 10 is explained below by reference to FIG. 2.

In the situation shown in FIG. 2, the computer 18 shown in FIG. 1 has been removed from the multi-purpose system 10 and has been replaced by a new computer 44. In this example, a hard disk 46 on the new computer 44 contains absolutely no data. In other words, the hard disk 46 has not yet been partitioned or formatted.

After the multi-purpose system 10 is switched on, the operating system is loaded into a working store in each of the computers 12, 14, 16 which is already in working order and the computers 12, 14, 16 are thereby put on stand-by. In the case of the new computer 44, the necessary data for doing this is missing from the unwritten hard disk 46. For this reason, a network card 48 in the new computer 44 here generates a signal conforming to a data protocol (BOOTP—Bootstrap Protocol) intended for the purpose. This signal is transmitted to the computer 12 which has the control system. In FIG. 2 this is indicated by an arrow for a transmission, T1.

The computer 12 thereupon transmits, in a transmission T2, an auxiliary operating system 50 to the new computer 44, which is then put in a position to provide simple functions by means of the auxiliary operating system 50. The program code of the auxiliary operating system here represents instructional data.

In addition, by means of an appropriate piece of server software (DHCPD—Dynamic Host Configuration Protocol Daemon), the computer 12 assigns to the new computer 44 a network address, which is here an IP address (IP—Internet Protocol) because the network facility 20 is a facility conforming to the Ethernet standard. The network address which is assigned to the new computer 44 is determined as a function of the connection 52 via which the new computer 44 is linked with the network facility 20. As a result, the new computer 44 in the example is given the same network address as had previously been given to the defective computer 18 which has been removed. The particular connection 52, 54, 56, 58, 60 to the network facility 20, to which the new computer 44 is connected, is notified to the computer 20 by a switch, not shown in FIG. 2, in the network facility 20.

The auxiliary operating system 50 requests from the file server 30 the hard disk image 38′ which had been stored by the computer 18 which was previously connected to the connection 52. The request for the transmission of the hard disk image 38′ is identified in. FIG. 2 as the transmission T3.

The file server 30 thereupon transmits the hard disk image 38′ to the new computer 44, which stores the hard disk image 38′ on its hard disk 46. The transmission procedure is identified in FIG. 2 as the transmission T4.

The storage of the hard disk image 38′ on the hard disk 46 automatically produces on the hard disk 46 a partition with a file system, and contained therein an operating system together with the additional software, as was also present on its hard disk 28 in the case of the defective computer 18. Hence, after a restart of the new computer 44 it makes available the same functions as did previously the computer 18. The integration of the new computer 44 into the multi-purpose system 10 is thereby completed.

In the case of the multi-purpose system 10, provision can be made for the partitions of the hard disks on all the computers 12, 14, 16, 44 to be overwritten simultaneously with each of their hard disk images, as stored on the file server 30. For this purpose, provision can be made for a user of the multi-purpose system 10 to select an appropriate function on the computer 12 to initiate the writing back of the hard disk images 32′, 34′, 36′, 38′ by the data server 30 to the hard disks of the computers. In this connection, provision can be made that there is a piece of software installed on each computer 12, 14, 16, 44, by means of which a computer is first restarted, or more precisely stated rebooted, and is then operated with an auxiliary operating system similar to the auxiliary operating system 50. For these auxiliary operating systems it is here possible to define in advance data about which hard disk image they should each call up from the data server 30 and should store on the hard disk of the computer concerned.

When all the hard disks have been rewritten with the appropriate hard disk images, the multi-purpose system 10 is restarted. The multi-purpose system 10 is then in the operating state as defined by the hard disk images 32′, 34′, 36′, 38′. All the changes to the software of the multi-purpose system 10, which had been made in the period between the creation of the hard disk images 32′, 34′, 36′, 38′ and the writing back of these to the hard disks of the computers from which they originated, are thereby undone. In particular, in the case that a malfunction of the multi-purpose system results from one of the changes, resetting the multi-purpose system enables its functional correctness to be reestablished rapidly and with little work.

In summary, the example shows that the invention enables the computers of the multi-purpose system to be rapidly matched to each other in such a way that the functional correctness of the multi-purpose system is guaranteed.

1.-12. (canceled)

13. A method for operating an apparatus comprising at least two interoperated electronic components, comprising:
copying data items for each of the electronic components in a configured state of the apparatus;
storing the copied data items outside of a storage device of the each of the electronic components;
storing the copied data items from the outside into the storage device of the each of the electronic components at a definable later point in time; and
determining the interoperation based on the copied data items.

14. The method as claimed in claim 13, further comprising:
detecting an operating state of the apparatus, and
interrupting the interoperation of the electronic components depending on the detected operating state, and
storing the copied data items into the storage device.

15. The method as claimed in claim 13, wherein the copied data items are stored in the storage device if an actuation of an operating element effects the electronic components.

16. The method as claimed in claim 13, wherein the copied data items are stored externally in a common central store.

17. The method as claimed in claim 13, wherein a storage image is created as a storage medium in the storage device for copying the data items.

18. The method as claimed in claim 13, wherein an operation mode of the electronic components is defined by conveying instructional data to the electronic components via a communication network.

19. The method as claimed in claim 18, wherein the electronic components store the copied data items in the storage device after conveying the instructional data to the electronic component.

20. The method as claimed in claim 13, wherein the copied data items or the instructional data comprises a program code.

21. The method as claimed in claim 13, wherein the copying of the data items and the storing of the copied data items are repeated at prescribed points in time.

22. The method as claimed in claim 13, wherein the copying of the data items and the storing of the copied data items are carried out after or before a change of the data items in the storage device.

23. A method for integrating an electronic component into an apparatus, comprising:
linking the electronic component to the apparatus;
storing data item of the electronic component defining an operation mode of the electronic component in a central storage device of the apparatus;
transmitting the data item from the central storage device to the electronic component; and
storing the data item in a storage device of the electronic component.

24. The method as claimed in claim 23, wherein the data item is copied from a storage device of another electronic component and is stored in the central storage device.

25. The method as claimed in claim 24, wherein the another electronic component is removed and replaced by the electronic component which is to be integrated into the apparatus.

26. The method as claimed in claim 13, wherein the data item comprises a program code.

27. A medical device, comprising:
a communication device;
an electronic component comprising a storage device that stores data item of the electronic component defining an operation mode of the electronic component;
a connection that links the electronic component to the communication device;
a central storage device that stores the data item; and
a control unit that transmits the data items from the central storage device to the electronic component.
